(19) United States
(12) Patent Application Publication
(10) Pub. No.: US 2017/0319686 A1
(43) Pub. Date: Nov. 9, 2017

(54) VACCINE AND PRIME-BOOST VACCINE

(71) Applicant: NIHON UNIVERSITY, Tokyo (JP)

(72) Inventors: Shihoko AIZAWA, Tokyo (JP); Mitsuo HONDA, Tokyo (JP); Satoshi HAYAKAWA, Tokyo (JP)

(21) Appl. No.: 15/526,809

(22) PCT Filed: Nov. 17, 2015
(86) PCT No.: PCT/JP2015/082258

§ 371 Date: May 15, 2017

(30) Foreign Application Priority Data
Nov. 17, 2014 (JP) 2014-232837

(51) Int. Cl. A61K 39/39; A61K 39/12; A61K 38/21; A61K 38/20; A61K 38/00; A61K 39/00

(52) U.S. Cl. CPC A61K 39/39; A61K 38/2013; A61K 39/12; A61K 38/00; A61K 38/212; A61K 2039/55511; A61K 39/00

(57) ABSTRACT

A vaccine for activating CD8 T cells including Bacillus Calmette-Guérin (BCG bacteria) which express a protein derived from a pathogenic microorganism as an active ingredient is beneficial because it can activate pathogenic microorganism-derived-antigen-specific CD8 T cells

### TECHNICAL FIELD

The present invention relates to a vaccine and a prime-boost vaccine.

Priority is claimed on Japanese Patent Application No. 2014-232837, filed Nov. 17, 2014, the content of which is incorporated herein by reference.

### BACKGROUND ART

Various severe infections including Ebola hemorrhagic fever, Crimean-Congo fever, South American hemorrhagic fever, Marburg disease, human immunodeficiency virus (HIV), and malaria in addition to tuberculosis are known.

Effective vaccines are not available for many of these severe infections. In addition, while Bacillus Calmette-Guérin (BCG) vaccines that are widely used as tuberculosis vaccines are known to have preventive effects for infantile tuberculosis and tuberculous meningitis, there is the viewpoint that they are ineffective at preventing adult pulmonary tuberculosis (for example, refer to Non-Patent Literature 1).

Therefore, more effective vaccines for infectious diseases are required.

### CITATION LIST

### Non-Patent Literature

### [Non-Patent Literature 1]

Andersen P., Tuberculosis vaccines—an update. Nat. Rev. Microbiol., 5(7), 484-7, 2007.

### SUMMARY OF INVENTION

### Technical Problem

Cell-mediated immunity is important for protection against infectious intracellular parasitic bacteria, such as Mycobacterium tuberculosis. In contrast, the inventors found that antigen-specific T cells activated according to immunization with the BCG vaccine are mainly CD4-positive T cells (helper T cells) and that there is hardly any activation of CD8-positive T cells (cytotoxic T cells) important for cell-mediated immunity.

Therefore, the present invention provides a vaccine capable of activating pathogenic microorganism-derived-antigen-specific CD8 T cells.

### Solution to Problem

The present invention is as follows.

(1) A vaccine for activating CD8 T cells including BCG bacteria which express a protein derived from a pathogenic microorganism as an active ingredient.
(2) The vaccine according to (1), wherein the BCG bacteria are BCG bacteria into which an expression vector of a gene that encode the protein has been introduced.
(3) The vaccine according to (2), wherein the expression vector is an expression vector capable of performing expression in both BCG bacteria and mammalian cells.
(4) The vaccine according to any one of (1) to (3), wherein the vaccine is a tuberculosis vaccine.
(5) The vaccine according to any one of (1) to (4), wherein the protein is Ag85B protein of Mycobacterium kansasii.
(6) A prime-boost vaccine including a combination of the vaccine according to any one of (1) to (5) and a DNA vaccine including an expression vector of the protein as an active ingredient, wherein prime immunization is performed using the vaccine according to any one of (1) to (5) and boost immunization is performed using the DNA vaccine.

### Advantageous Effects of Invention

According to the present invention, it is possible to provide a vaccine capable of activating pathogenic microorganism-derived-antigen-specific CD8 T cells.

### BRIEF DESCRIPTION OF DRAWINGS

FIG. 1A is a graph showing abundances of interferon-γ-producing cells in the entire mononuclear cell group.

FIG. 1B is a graph showing abundances of interferon-γ-producing cells in CD4-positive T cell-removed groups and CD8-positive T cell-removed groups.

FIG. 1C is a graph showing abundances of interferon-γ-producing cells in CD4-positive T cell and CD8-positive T cell-removed groups.

FIG. 2A is a diagram showing a structure of a p3HA85B vector.

FIG. 2B is a diagram showing a structure of a pDNA85B vector.

FIG. 3 is a picture showing results of western blotting by which expression of Ag85B proteins is examined.

FIG. 4A is a picture showing results of western blotting of BCG bacteria.

FIG. 4B is a picture showing results of western blotting of 293T cells.

FIG. 5 shows graphs of results of flow cytometry.

FIG. 6 is a graph showing abundances of CD4-positive T cells that produce cytokines.

FIG. 7A is a graph showing results obtained by analyzing CD4-positive T cells that produce cytokines in further detail.

FIG. 7B is a pie chart of the results of FIG. 7A.

FIG. 8 is a graph showing abundances of CD8-positive T cells that produce cytokines.

FIG. 9 is a graph of the summarized results of intracellular cytokine analysis.

FIG. 10A is a graph of the summarized results of intracellular cytokine analysis.

FIG. 10B is a graph of the summarized results of intracellular cytokine analysis.

### DESCRIPTION OF EMBODIMENTS

In one embodiment, the present invention provides a vaccine for activating CD8 T cells including BCG bacteria which express a protein derived from a pathogenic microorganism as an active ingredient.

As will be described below, according to the vaccine of the present embodiment, it is possible to activate (induce) pathogenic microorganism-derived-antigen-specific CD8 T cells. Activation of CD8-positive T cells means that, when the above protein derived from the pathogenic microorganism are exposed to an (immunized) living body to which a vaccine is administered, CD8-positive T cells that produce specific cytokines are formed. Therefore, the vaccine of the present embodiment is also referred to as a CD8-positive T cell activator. As the above cytokines, for example, interferon-γ, interleukin-2, TNF-α, and MIP-1α may be exemplified.

### (Pathogenic Microorganisms)

Pathogenic microorganisms targeted by the vaccine of the present embodiment are not particularly limited. Since the vaccine of the present embodiment can activate pathogenic microorganism-derived-antigen-specific CD8 T cells, it can also be applied to pathogenic microorganisms for which it is considered difficult to obtain an effective vaccine.

Therefore, the pathogenic microorganisms may be pathogenic microorganisms for which it is considered difficult to obtain an effective vaccine, and for example, Mycobacterium tuberculosis, which is a tuberculosis pathogen, Ebola hemorrhagic fever viruses, which are causative viruses of Ebola hemorrhagic fever, Crimean-Congo viruses, which are causative viruses of Crimean-Congo fever, Junin viruses, which are causative viruses of South American hemorrhagic fever, arenaviridae viruses such as Sabia virus, Guanarito virus, and Machupo virus, Marburg viruses, which are causative viruses of Marburg disease, malaria parasites, which are causative microorganisms of malaria, and affiliated microorganisms of the above pathogenic microorganisms, for example, attenuated strains, may be exemplified.

In the vaccine of the present embodiment, as proteins derived from pathogenic microorganisms expressed in BCG bacteria, for example, proteins showing a high expression level when there is infection with the above pathogenic microorganisms may be exemplified. More specifically, Ag85B proteins of Mycobacterium tuberculosis, Ag85B proteins of Mycobacterium kansasii, which are acid-fast bacteria the same as Mycobacterium tuberculosis, envelope glycoproteins of Ebola hemorrhagic fever viruses, gp140 proteins of HIV viruses, and malaria antigen proteins of malaria parasites may be exemplified.

According to the vaccine of the present embodiment, by changing the protein derived from pathogenic microorganisms and expressed in BCG bacteria, it is possible to easily provide a vaccine for multifarious pathogenic microorganisms. For example, BCG bacteria in which Ag85B protein of Mycobacterium kansasii are expressed can be used as a tuberculosis vaccine capable of activating CD8 T cells.

In the vaccine of the present embodiment, BCG bacteria are used as hosts in which a protein derived from a pathogenic microorganism is expressed. BCG bacteria have been successfully inoculated into humans as a vaccine for a long time and the safety thereof is proven. Therefore, according to the vaccine of the present embodiment, it is possible to conveniently provide a safe vaccine.

In addition, in the vaccine of the present embodiment, when BCG bacteria in which a protein derived from a pathogenic microorganism is expressed are used, it is possible to activate pathogenic microorganism-derived-antigen-specific CD8 T cells. On the other hand, if a protein derived from a pathogenic microorganism is simply administered as a vaccine, it is difficult to effectively activate antigen specific CD8-positive T cells.

The inventors speculated that CD8-positive T cells can be activated when the vaccine of the present embodiment is administered because, in order to activate CD8-positive T cells, it is important that BCG bacteria in which a protein derived from a pathogenic microorganism is expressed are phagocytosed by phagocytes such as macrophages in a living body and a protein derived from a pathogenic microorganism is appropriately processed in cells and antigen presentation is performed.

Furthermore, as will be described below, according to the vaccine of the present embodiment, it is possible to induce CD8-positive T cells that have an ability for one cell to produce a plurality of kinds of cytokine, that is, polyfunctionality. Since T cells having polyfunctionality have an excellent infection protective ability, it is possible to provide a high infection protective ability according to the vaccine of the present embodiment.

### (Expression Vector)

Expression of a protein derived from a pathogenic microorganism using BCG bacteria as hosts is preferably performed by introducing an expression vector of a gene that encode a protein derived from a pathogenic microorganism into BCG bacteria. Therefore, it is possible to easily express a protein derived from a pathogenic microorganism using BCG bacteria as hosts. In addition, when an appropriate promoter is selected or an enhancer is combined, it is possible to increase an expression level of a protein derived from a pathogenic microorganism.

In addition, an expression vector that is maintained in the form of a plasmid in BCG bacteria is preferable rather than an expression vector that is introduced into genomes of BCG bacteria. Therefore, since the quality of BCG bacteria in which a protein derived from a pathogenic microorganism is expressed is stabilized, quality control of the vaccine of the present embodiment becomes easier.

As a promoter for expressing a protein in BCG bacteria, for example, pAL5000 derived from Mycobacterium fortuitum may be exemplified.

The above expression vector is more preferably an expression vector capable of performing expression in both BCG bacteria and mammalian cells. BCG bacteria administered to a living body are phagocytosed and destroyed by phagocytes such as macrophages. Therefore, when the expression vector released due to the destruction of BCG bacteria can be expressed in both BCG bacteria and mammalian cells, expression of a protein derived from a pathogenic microorganism continues also in cells such as macrophages. As a result, more proteins derived from the pathogenic microorganism are supplied into a living body, and it is possible to achieve immunization more effectively.

The vaccine of the present embodiment is preferably intradermally administered. In addition, a dose may be appropriately adjusted depending on an administration subject and may be, for example, about 2×10⁶ colony-forming units (CFU) for each mouse.

### (Prime-Boost Vaccine)

In one embodiment, the present invention provides a prime-boost vaccine which includes a combination of the vaccine and a DNA vaccine including an expression vector of a protein that is expressed in BCG bacteria serving as an active ingredient of the vaccine as an active ingredient, wherein the vaccine is used to perform prime immunization and the DNA vaccine is used to perform boost immunization.

The prime-boost vaccine is a combination of a vaccine used for prime immunization and another vaccine that performs boost immunization one or more times or a method of administering such a vaccine.

In addition, the DNA vaccine is a vaccine that is translated into a protein when administered to a living body and the protein contribute to achievement of immunization.

In the prime-boost vaccine of the present embodiment, prime immunization is performed using recombinant BCG bacteria in which Ag85B protein is expressed and boost immunization is performed using a DNA vaccine including an expression vector of Ag85B protein as an active ingredient.

The recombinant BCG bacteria are preferably intradermally administered. In addition, a dose may be appropriately adjusted depending on an administration subject and may be, for example, about 2×10⁶ CFU for each mouse.

In addition, the DNA vaccine is preferably intramuscularly injected. In addition, a dose may be appropriately adjusted depending on an administration subject, and may be, for example, about 100 μg for each mouse.

A vaccine administration schedule may be appropriately adjusted. For example, a schedule in which first boost immunization with a DNA vaccine is performed 3 weeks after prime immunization is performed using the recombinant BCG bacteria, second boost immunization is performed using a DNA vaccine after another 3 weeks, and third boost immunization is performed using a DNA vaccine after another 2 weeks may be exemplified.

### Other Embodiments

In one embodiment, the present invention provides BCG bacteria expressing a protein derived from a pathogenic microorganism for activating pathogenic microorganism-derived-antigen-specific CD8 T cells.

In one embodiment, the present invention provides a use of BCG bacteria expressing a protein derived from a pathogenic microorganism, for the manufacture of a vaccine for activating pathogenic microorganism-derived-antigen-specific CD8 T cells.

In one embodiment, the present invention provides a method of activating pathogenic microorganism-derived-antigen-specific CD8 T cells, including administering an effective amount of BCG bacteria expressing a protein derived from a pathogenic microorganism to a patient in need thereof.

In one embodiment, the present invention provides a method of activating pathogenic microorganism-derived-antigen-specific CD8 T cells. The method includes a process in which a vaccine including BCG bacteria expressing a protein derived from a pathogenic microorganism as an active ingredient is administered to perform prime immunization and a process in which a DNA vaccine including an expression vector of the protein as an active ingredient is administered to perform boost immunization.

### Examples

Next, the present invention will be described in further detail with reference to experimental examples, but the present invention is not limited to the following experimental examples.

### Experimental Example 1

Guinea pigs were immunized with BCG bacteria and it was examined whether purified tuberculin (purified protein derivatives, hereinafter referred to as “PPDs”)-specific CD4-positive T cells and PPD-specific CD8-positive T cells were induced.

### (Immunization)

2×10⁶ CFU/mouse of BCG bacteria was intradermally administered to guinea pigs (Japan SLC, Inc.) and blood samples were taken and examined 12 weeks after immunization.

### (Detection of Interferon-γ-Producing Cells Using an ELISPOT Method)

The collected blood was centrifuged by density gradient centrifugation using a blood cell separation solution (trade name “Lymphosepar,” TAKARA BIO Inc.) and peripheral blood mononuclear cells were collected. Next, a cell-based enzyme-linked immunospot (ELISPOT) assay was used to measure a proportion of interferon-γ-producing cells in the collected peripheral blood mononuclear cells.

Specifically, first, the collected peripheral blood mononuclear cells were divided into four groups. The first group (the entire mononuclear cell group) was used in that state in the next operation. The second group (CD4-positive T cell-removed group) from which CD4-positive T cells were removed was used in the next operation. The third group (CD8-positive T cell-removed group) from which CD8-positive T cells were removed was used in the next operation. The fourth group (CD4-positive T cell and CD8-positive T cell-removed group) from which CD4-positive T cells and CD8-positive T cells were removed was used in the next operation. CD4-positive T cells or CD8-positive T cells were removed using a cell sorter.

Next, 1 μg/mL of PPDs was added to mediums of each group of the cells and stimulated, and this was used in the next operation after 20 hours. As a negative control, groups in which phosphate-buffered saline (PBS) was added in place of PPDs were prepared. In addition, as a positive control, groups in which 1 μg/mL of concanavalin A (ConA) was added in place of PPDs were prepared.

Next, the mediums and the groups of the cells were added to a 96-well microtiter plate with a PVDF membrane bottom to which anti-interferon-γ antibodies were bound and incubated for 5 hours. As a result, when the cells secreted interferon-γ, the secreted interferon-γ was bound to anti-interferon-γ antibodies present in the vicinity of cells. Then, cells were washed and interferon-γ bound on the membrane was detected by immunostaining. As a result, spots indicating the presence of interferon-γ were formed in a part of the membrane in which interferon-γ-producing cells were present. One spot corresponded to one interferon-γ-producing cell. Therefore, when the number of spots was measured, it was possible to measure an abundance of interferon-γ-producing cells.

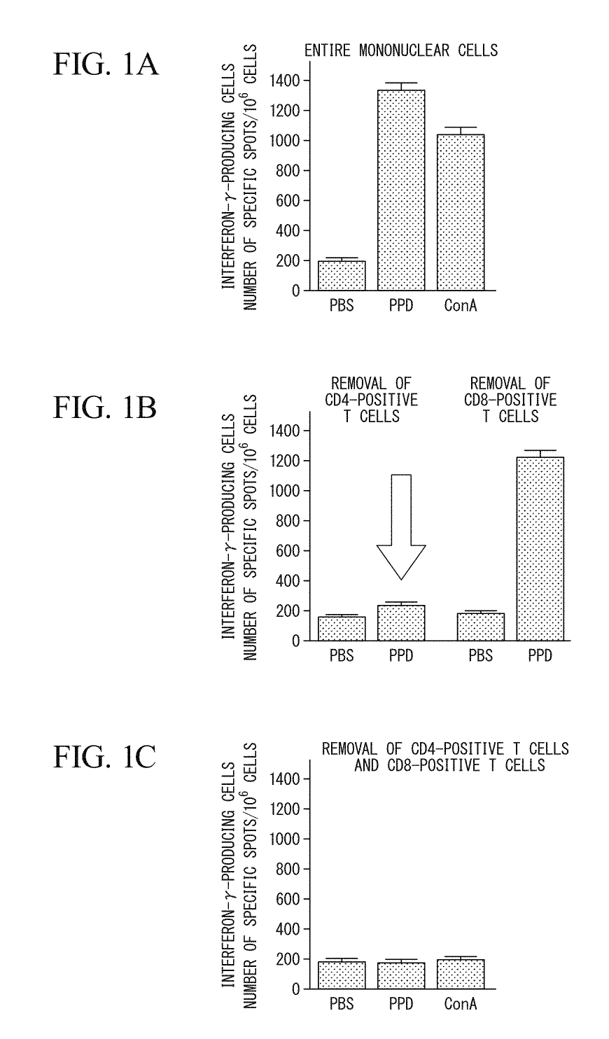

FIG. 1A is a graph showing abundances of interferon-γ-producing cells in the entire mononuclear cell group. FIG. 1B is a graph showing abundances of interferon-γ-producing cells in CD4-positive T cell-removed groups and CD8-positive T cell-removed groups. FIG. 1C is a graph showing abundances of interferon-γ-producing cells in CD4-positive T cell and CD8-positive T cell-removed groups.

As shown in FIG. 1A, in the entire mononuclear cell group, when stimulation was performed with PPDs, cells producing interferon-γ were induced. In addition, as shown in FIG. 1B, when CD4-positive T cells were removed, there were significantly fewer cells producing interferon-γ due to stimulation with PPDs. In addition, when CD8-positive T cells were removed, the number of cells producing interferon-γ due to stimulation with PPDs hardly changed. In addition, as shown in FIG. 1C, when CD4-positive T cells and CD8-positive T cells were removed, there were significantly fewer cells producing interferon-γ due to stimulation with PPDs.

Based on the above results, it can be clearly understood that PPD-specific interferon-γ-producing cells induced as a result of immunization with BCG bacteria were mainly CD4-positive T cells and PPD-specific interferon-γ-producing CD8-positive T cells were induced less.

### Experimental Example 2

An expression vector of Ag85B protein of Mycobacterium kansasii was prepared.

(p3HA85B Vector)

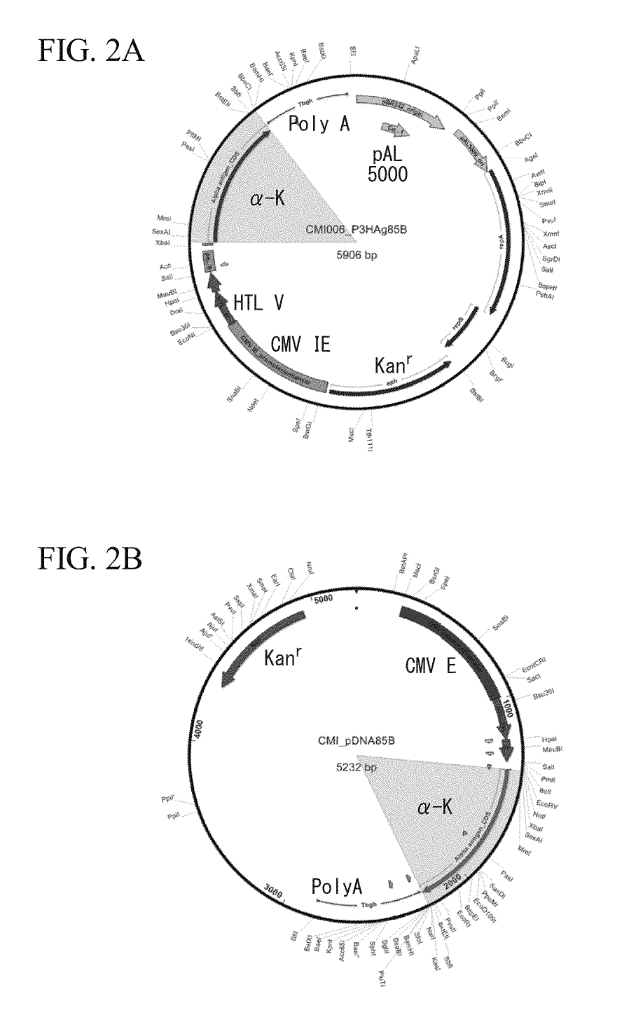

p3HA85B, which is an expression vector capable of expressing Ag85B protein in both BCG bacteria and mammalian cells, was prepared. p3HA85B had a pAL5000 promoter operable in BCG bacteria upstream of genes (SEQ ID NO. 1) that encode Ag85B protein of Mycobacterium kansasii and had a cytomegalovirus (CMV) promoter and an HTLV-1 promoter operable in mammalian cells upstream of the pAL5000 promoter. Furthermore, a polyA signal gene was incorporated downstream of genes that encode Ag85B protein. FIG. 2A shows a structure of the p3HA85B vector.

(pDNA85B vector)

pDNA85B, which is an expression vector capable of expressing Ag85B protein in mammalian cells, was prepared. pDNA85B had a cytomegalovirus (CMV) promoter operable in mammalian cells upstream of genes (SEQ ID NO. 1) that encode Ag85B protein. FIG. 2B shows a structure of the pDNA85B vector.

### Experimental Example 3

The expression vector p3HA85B was introduced into BCG bacteria and BCG bacteria in which Ag85B protein of Mycobacterium kansasii was expressed were prepared. Next, western blotting was performed to determine the expression of Ag85B protein.

(Introduction of p3HA85B into BCG Bacteria)

The expression vector p3HA85B was introduced into BCG bacteria by an electroporation method. Next, using kanamycin resistance as an index, BCG bacteria into which p3HA85B had been introduced were selected and cloned to prepare BCG bacteria in which Ag85B protein was expressed.

### (Western Blotting)

BCG bacteria in which Ag85B protein was expressed and general BCG bacteria serving as a control were dissolved in Cell Lysis Buffer (Cell Signaling Technology).

Next, an RC DC protein assay (Bio-rad) was used to quantify a protein content and the same amounts of protein was electrophoresed in 4-12% Bis-Tris gel (Life Technologies).

Next, the electrophoresed protein was transferred to a PVDF membrane (Life Technologies) and immunostained with anti-Ag85B antibodies. Horseradish peroxidase (HRP)-labeled anti-rabbit IgG antibodies (Cell Signaling Technology) were used as secondary antibodies. An Amersham ECL Western Blotting Analysis System (GE Healthcare) was used to detect a signal.

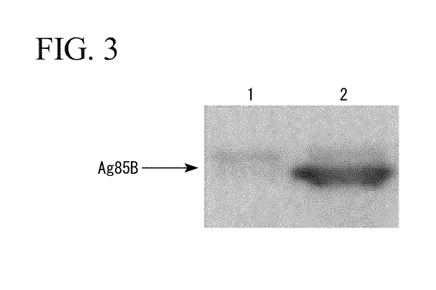

FIG. 3 is a picture showing results of western blotting. Lane 1 shows results of BCG bacteria serving as a control. Lane 2 shows results of BCG bacteria in which Ag85B protein was expressed. Moreover, anti-Ag85B antibodies used here were bound to both Ag85B protein of BCG bacteria and Ag85B protein of Mycobacterium kansasii.

As a result, it was confirmed that an expression level of Ag85B protein in BCG bacteria increased by a factor of 50 to 100 due to introduction of the expression vector p3HA85B.

### Experimental Example 4

It was examined whether it was possible to express Ag85B protein in mammalian cells using the expression vector p3HA85B.

p3HA85B was introduced and expressed in 293T cells, which are human embryonic kidney epithelial cells, using a gene introduction reagent (trade name “Lipofectamin 2000,” Life Technologies).

Next, in the same manner as in Experimental Example 3, western blotting was performed to determine the expression of Ag85B protein. As a positive control, western blotting of BCG bacteria in which Ag85B protein was expressed in Experimental Example 3 was performed.

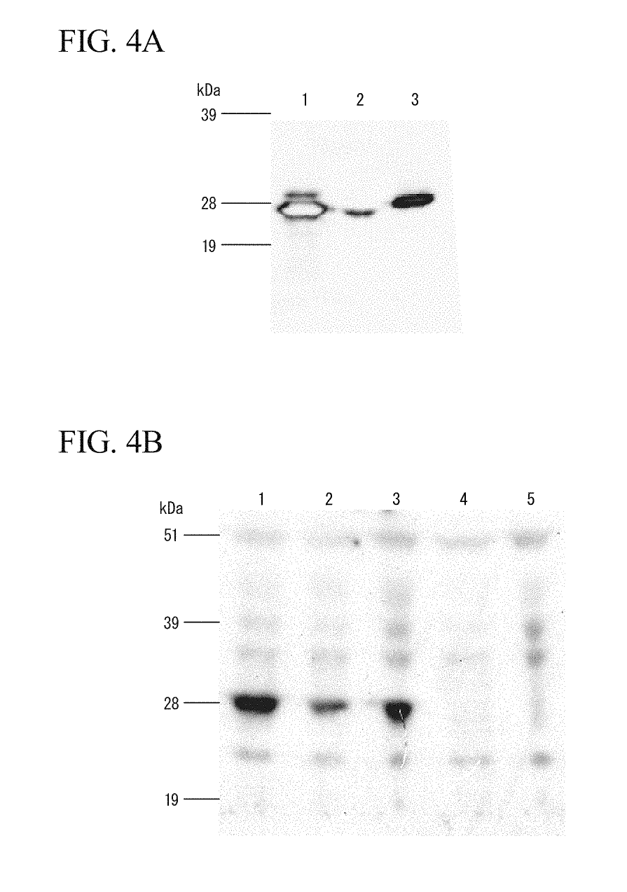

FIG. 4A is a picture showing results of western blotting of BCG bacteria serving as a positive control. Lane 1 shows results of BCG bacteria serving as a positive control. In addition, Lane 2 shows results obtained by providing recombinant Ag85B protein for western blotting. In addition, Lane 3 shows results obtained by providing recombinant MPB59 protein, which are known as widely cross-reactive protein for western blotting.

FIG. 4B is a picture showing results of western blotting of 293T cells. Lane 3 shows results of 293T cells into which the expression vector p3HA85B was introduced. Lane 4 shows results of 293T cells into which only an empty vector p3H8.3 was introduced as a negative control. Lane 5 shows results obtained by providing only PBS for western blotting as a negative control. In addition, Lane 1 and Lane 2 show results of 293T cells into which the pDNA85B prepared in Experimental Example 2 was introduced.

As a result, it was confirmed that it was possible to express Ag85B protein in both BCG bacteria and mammalian cells according to introduction of the p3HA85B vector. In addition, it was confirmed that it was possible to express Ag85B protein in mammalian cells using the pDNA85B vector.

### Experimental Example 5

Mice were immunized with BCG bacteria and it was examined whether antigen-specific CD8-positive T cells were induced.

### (Immunization)

2×10⁶ CFU/mouse of BCG bacteria was intradermally administered to C57BL/6J mice (Oriental Yeast Co., Ltd.). Dissection and examination were performed 3 weeks after immunization.

### (Intracellular Cytokine Analysis)

Spleen cells were isolated from the above immunized mice and antigenically stimulated in an RPMI-1640 medium to which fetal bovine serum 10 v/v %, 2-mercaptoethanol, DNase 1 U/mL, penicillin and streptomycin were added for 6 hours. 10 μg/mL of PPDs was used as antigens.

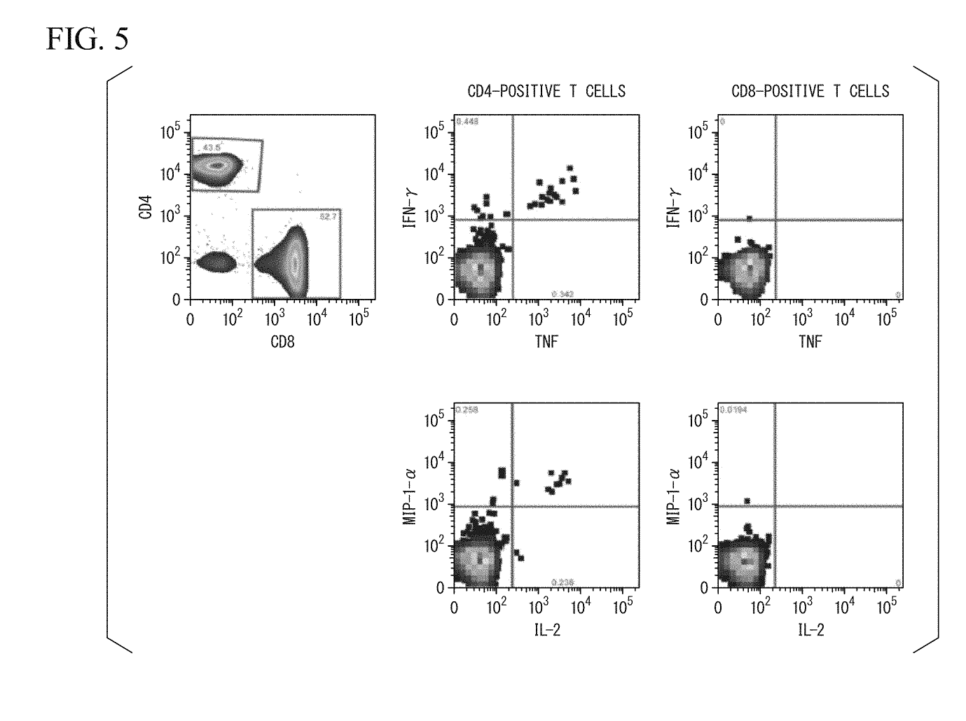

Next, cells were collected and cell surfaces were stained with APC-labeled anti-CD3 antibodies (BD biociences), PE-cy7-labeled anti-CD4 antibodies (BioLegend), and PerCP cy5.5-labeled anti-CD8 antibodies (BD biociences). Further, the cells were treated using a BD Cytofix/Cytoperm Fixation/Permeabilization Solution Kit (BD biociences), and intracellular cytokines were stained with Alexa Fluor 488-labeled anti-TNF-α antibodies (BD biociences), PE-labeled anti-interferon-γ antibodies (BD biociences), APC-Cy7-labeled anti-interleukin-2 antibodies (BD biociences), and V500-labeled anti-MIP-1α antibodies (BD biociences). The stained cells were analyzed using FACSverse software (BD biociences) and FlowJo software (TreeStar). FIG. 5 shows graphs of results of flow cytometry.

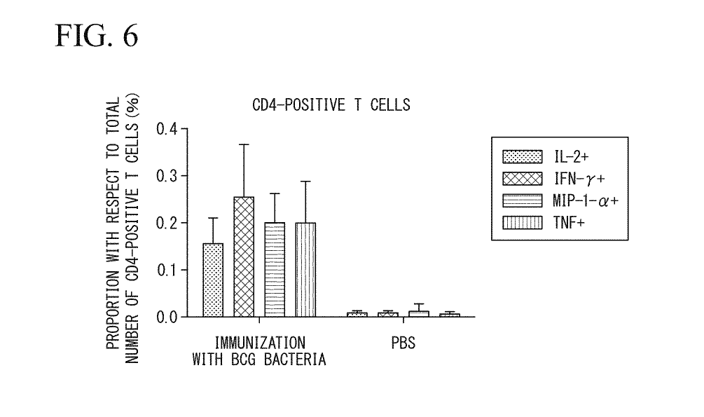

FIG. 6 is a graph showing abundances of CD4-positive T cells that produce cytokines. Cells stimulated in phosphate-buffered saline (PBS) in place of PPDs were used as a negative control. The result was that CD4-positive T cells were induced according to immunization with BCG bacteria.

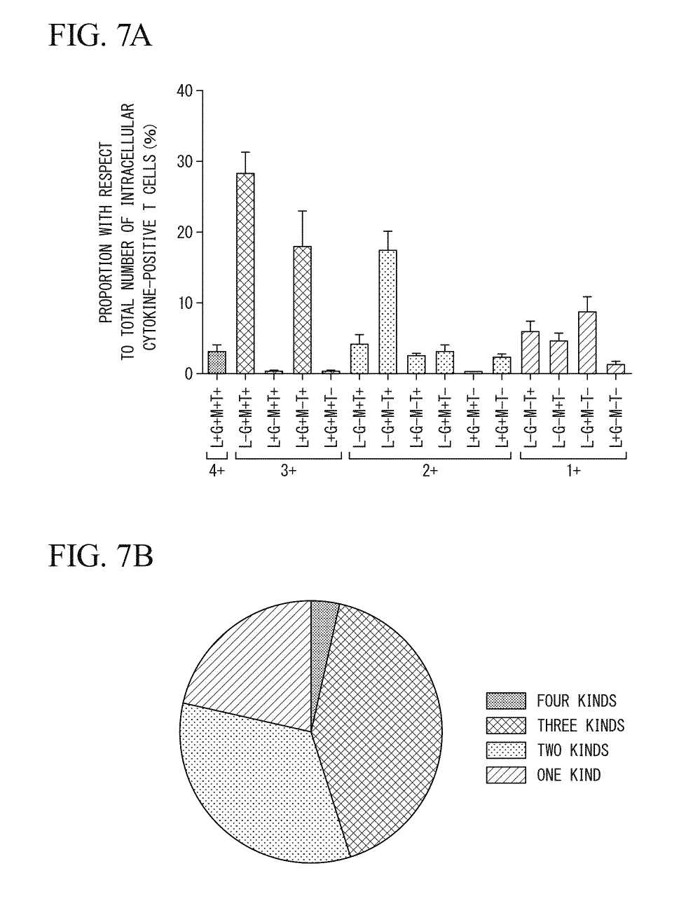

FIG. 7A is a graph showing results obtained by analyzing CD4-positive T cells that produce cytokines in further detail. In FIG. 7A, L represents interleukin-2, G represents interferon-γ, M represents MIP-1α, and T represents TNF-α. In addition, “+” represents positive and “−” represents negative. Regarding the results, it can be clearly understood that one CD4-positive T cell produces one to four kinds of cytokine. That is, polyfunctional CD4-positive T cells were induced according to immunization with BCG bacteria. In addition, FIG. 7B shows a pie chart of the results of FIG. 7A.

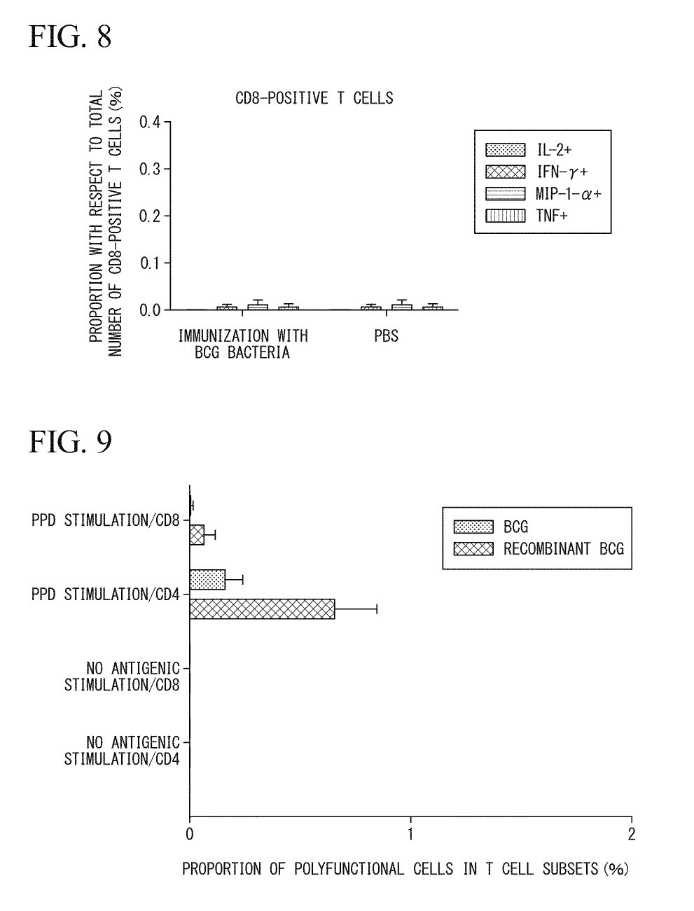

FIG. 8 is a graph showing abundances of CD8-positive T cells that produce cytokines. Cells stimulated in phosphate-buffered saline (PBS) in place of PPDs were used as a negative control. The result was that hardly any CD8-positive T cells were induced according to immunization with BCG bacteria.

Based on the above results, it can be clearly understood that CD4-positive T cells were induced according to immunization with BCG bacteria and hardly any CD8-positive T cells were induced. In addition, it was confirmed that the induced CD4-positive T cells were polyfunctional.

### Experimental Example 6

Mice were immunized with BCG bacteria or BCG bacteria into which the expression vector p3HA85B was introduced and it was examined whether polyfunctional CD8-positive T cells were induced.

### (Immunization)

2×10⁶ CFU/mouse of BCG bacteria or BCG bacteria into which the expression vector p3HA85B was introduced were intradermally administered to BALB/c mice (Oriental Yeast Co., Ltd.). Dissection and examination were performed 3 weeks after immunization.

### (Intracellular Cytokine Analysis)

Spleen cells were isolated from the above immunized mice and antigenically stimulated in an RPMI-1640 medium to which fetal bovine serum 10 v/v %, 2-mercaptoethanol, DNase 1 U/mL, penicillin and streptomycin had been added for 24 hours. 10 μg/mL of PPDs were used as antigens. Cells not subjected to antigenic stimulation were used as a negative control.

Next, the cells were collected and intracellular cytokine analysis was performed through flow cytometry in the same manner as in Experimental Example 5.

FIG. 9 is a graph of the summarized analysis results. In FIG. 9, “recombinant BCG” refers to a group that was immunized with BCG bacteria into which the expression vector p3HA85B was introduced. The result was that, when no antigenic stimulation was performed, the presence of polyfunctional CD4-positive T cells and polyfunctional CD8-positive T cells was not observed. In addition, the presence of polyfunctional CD8-positive T cells as a result of antigenic stimulation was not observed in a group immunized with BCG bacteria. On the other hand, the presence of polyfunctional CD8-positive T cells was observed in a group immunized with BCG bacteria into which the expression vector p3HA85B was introduced.

In addition, as a result of antigenic stimulation, the presence of polyfunctional CD4-positive T cells was observed in both a group immunized with BCG bacteria and a group immunized with BCG bacteria into which the expression vector p3HA85B was introduced.

Based on the above results, it can be clearly understood that polyfunctional CD8-positive T cells could be induced according to immunization using BCG bacteria into which the expression vector p3HA85B was introduced.

### Experimental Example 7

Mice were immunized with a prime-boost vaccine in which prime immunization was performed using BCG bacteria into which the expression vector p3HA85B was introduced, boost immunization was performed using the pDNA85B vector (DNA vaccine), and inducing of polyfunctional CD8-positive T cells was examined. Mice immunized with only the pDNA85B vector were used as a control.

Specifically, BCG bacteria into which the expression vector p3HA85B was introduced were intradermally administered to BALB/c mice (Oriental Yeast Co., Ltd.) and C57BL/6J mice (Oriental Yeast Co., Ltd.) at 2×10⁶ CFU per mouse for prime immunization. Next, after 3 weeks, the pDNA85B vector was intramuscularly injected at 100 μg per mouse for first boost immunization. After another 3 weeks, the pDNA85B vector was intramuscularly injected at 100 μg per mouse for second boost immunization. After another 2 weeks, the pDNA85B vector was intramuscularly injected at 100 μg per mouse for third boost immunization. Dissection and examination were performed 3 weeks after third boost immunization.

Mice immunized with only the pDNA85B vector were used as subjects. Specifically, the pDNA85B vector was intramuscularly injected at 100 μg per mouse for prime immunization. Next, after 3 weeks, the pDNA85B vector was intramuscularly injected at 100 μg per mouse for first boost immunization. After another 3 weeks, the pDNA85B vector was intramuscularly injected at 100 μg per mouse for second boost immunization. After another 2 weeks, the pDNA85B vector was intramuscularly injected at 100 μg per mouse for third boost immunization. Dissection and examination were performed 3 weeks after third boost immunization.

### (Intracellular Cytokine Analysis)

Spleen cells were isolated from the above immunized mice and antigenically stimulated in an RPMI-1640 medium to which fetal bovine serum 10 v/v %, 2-mercaptoethanol, DNase 1 U/mL, penicillin and streptomycin had been added for 24 hours. Peptide Pools (Pool 1 to Pool 6) of Ag85B protein shown in the following Table 1 were used as antigens. The peptide pools were used at a concentration of 2.5 μg/mL when antigenic stimulation was performed. In addition, cells not subjected to antigenic stimulation were used as a negative control. Next, the cells were collected and intracellular cytokine analysis was performed through flow cytometry in the same manner as in Experimental Example 5.

TABLE 1

| Pool | Content of pool |
|---|---|
| Pool 1 | A peptide pool of amino acid sequences of residues 41 to 55, 46 |
| | to 60, 51 to 65, 56 to 70, 61 to 75, 66 to 80, 71 to 85, 76 to 90, |
| | 81 to 95, and 86 to 100 in amino acid sequence (SEQ ID NO. 2) |
| | of Ag85B protein |
| Pool 2 | A peptide pool of amino acid sequences of residues 91 to 105, |
| | 96 to 110, 101 to 115, 106 to 120, 111 to 125, 116 to 130, 121 |
| | to 135, 126 to 140, 131 to 145, and 136 to 150 in amino acid |
| | sequences (SEQ ID NO. 2) of Ag85B protein |
| Pool 3 | A peptide pool of amino acid sequences of residues 141 to 155, |
| | 146 to 160, 151 to 165, 156 to 170, 161 to 175, 166 to 180, 171 |
| | to 185, 176 to 190, 181 to 195, and 186 to 200 in amino acid |
| | sequences (SEQ ID NO. 2) of Ag85B protein |
| Pool 4 | A peptide pool of amino acid sequences of residues 191 to 205, |
| | 196 to 210, 201 to 215, 206 to 220, 211 to 225, 216 to 230, 221 |
| | to 235, 226 to 240, 231 to 245, and 236 to 250 in amino acid |
| | sequences (SEQ ID NO. 2) of Ag85B protein |
| Pool 5 | A peptide pool of amino acid sequences of residues 241 to 255, |
| | 246 to 260, 250 to 265, 256 to 270, 261 to 275, 266 to 280, 271 |
| | to 285, 276 to 290, 281 to 295, and 286 to 300 in amino acid |
| | sequences (SEQ ID NO. 2) of Ag85B protein |
| Pool 6 | A peptide pool of amino acid sequences of residues 291 to 305, |
| | 296 to 310, 300 to 315, 306 to 320, and 311 to 325 in amino |
| | acid sequences (SEQ ID NO. 2) of Ag85B protein |

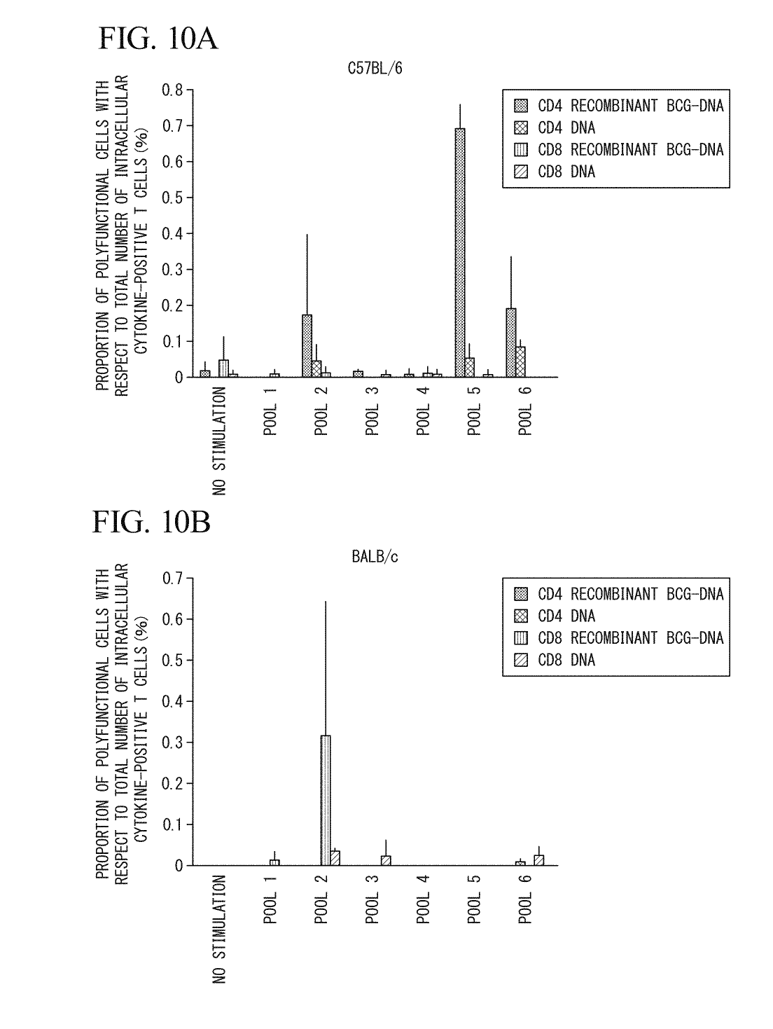

FIG. 10A is a graph of the summarized analysis results for C57BL/6J mice. FIG. 10B is a graph of the summarized analysis results for BALB/c mice. In FIGS. 10A and 10B, “recombinant BCG-DNA” refers to a group (prime-boost immunized group) on which prime immunization was performed using BCG bacteria into which the expression vector p3HA85B was introduced and boost immunization was performed using the pDNA85B vector. In addition, “DNA” refers to a group immunized with only the pDNA85B vector.

The result was that, when antigenic stimulation was not performed in either spleen cells of C57BL/6J mice or BALB/c mice, the presence of polyfunctional CD4-positive T cells and polyfunctional CD8-positive T cells was barely observed.

In addition, when antigenic stimulation was performed in spleen cells of C57BL/6J mice, the prime-boost immunized mice had a higher abundance of polyfunctional CD4-positive T cells than the mice immunized with only the pDNA85B vector (such as Pools 2, 5, and 6).

In addition, when antigenic stimulation was performed in spleen cells of BALB/c mice, the prime-boost immunized mice had a higher abundance of polyfunctional CD8-positive T cells than the mice immunized with only the pDNA85B vector (such as Pool 2).

### INDUSTRIAL APPLICABILITY

According to the present invention, it is possible to provide a vaccine capable of activating antigen-specific CD8-positive T cells derived from pathogenic microorganisms.

1. A vaccine for activating CD8 T cells comprising Bacillus Calmette-Guérin (BCG bacteria) which express a protein derived from a pathogenic microorganism as an active ingredient.

2. The vaccine according to claim 1,
wherein the BCG bacteria are BCG bacteria into which an expression vector of a gene that encode the protein has been introduced.

3. The vaccine according to claim 2,
wherein the expression vector is an expression vector capable of performing expression in both BCG bacteria and mammalian cells.

4. The vaccine according to claim 1,
wherein the vaccine is a tuberculosis vaccine.

5. The vaccine according to claim 1,
wherein the protein is Ag85B protein of Mycobacterium kansasii.

6. A prime-boost vaccine comprising, a combination of the vaccine according to claim 1 and a DNA vaccine including an expression vector of the protein as an active ingredient,
wherein prime immunization is performed using the vaccine according to claim 1 and boost immunization is performed using the DNA vaccine.

7. A method of activating pathogenic microorganism-derived-antigen-specific CD8 T cells in a patient in need thereof, comprising a step of administering an effective amount of BCG bacteria expressing a protein derived from a pathogenic microorganism to the patient.

8. The method according to claim 7, further comprising a step of administering a DNA vaccine including an expression vector of the protein as an active ingredient to the patient.


SEQUENCE LISTING (2 sequences)

SEQ ID NO: 1 - DNA, 1396 bp, Mycobacterium kansasii
  misc_feature (270)..(1244): Ag85B CDS
  misc_feature (270)..(389): signal peptide
  misc_feature (390)..(1244): mature Ag85B
gttaactattctttgtaccgctccccgcctgccgccttctgccctgctccgggtgcatagcacccgtttgcgctccggattatccgggccgcaacggggcaacgggggaagcggtggagtccgtcgccgactcgcatagcaccgttgctgtgttggcgggggtaaccgatatcgaaatggaatgacttcgcgtcccgatcgacatttgccctactcacacggtaagttctgccgggagcacgcgagcacatacggacaaggggcagggtatgacagacgtgagcgggaagattcgggcgtggggccgacgccttctggtcggcgcggccgctgctgcggcccttcctggcctggtcggactcgccggcggagcggcgaccgcgggagcgttctcccgtcccggcctgccggtggagtacctccaggtgccgtcggctgcgatgggtcgcagtatcaaggttcaattccaaagtggcggggacaactcgccggcggtgtacctgctcgacggtctccgcgctcaagacgactacaacggctgggacatcaacaccccggccttcgagtggtactaccaatcgggcctgtcggtcatcatgccggtcggcggacagtccagtttctacagtgactggtacagcccggcctgcggcaaggccggctgcacgacctacaagtgggagaccttcctgaccagcgagctgccgcaatggctgtccgcgaaccggagtgtcaagcccaccggaagcgccgcggtcggcatctcgatggccggctcgtcggccctgatcctgtccgtctaccacccgcagcagttcatctacgcgggttcgttgtcggccctgatggacccctcccaggggatggggccgtctctgatcggcttggcgatgggtgacgccggtggttacaaggcctcggacatgtggggaccctcgagtgacccagcctggcagcgtaacgacccgtcgctgcacattccggagctggtcgccaacaacacccgcctgtggatctactgcggcaacggcaccccgtccgagttgggcggtgccaatgttccggccgaattcctggagaacttcgttcgcagcagcaacctgaaattccaggacgcctacaacgccgcgggcggccacaacgccgtgttcaatttggacgccaacggaacgcacagctgggagtactggggcgcgcagctcaacgccatgaagggtgacctgcaggccagcctgggcgcccgctgatcgcgcaacggttgccgctactgggcttgacggcaagacgccgtcaagccagtagtgtgttcggcaccttgaacgctggtccgccatgttcaacgagccggtctacctgcccgcaccgaacaagctggtgacatcgacccatgcggtgc

SEQ ID NO: 2 - protein, 325 aa, Mycobacterium kansasii
  misc_feature: Ag85B
MTDVSGKIRAWGRRLLVGAAAAAALPGLVGLAGGAATAGAFSRPGLPVEYLQVPSAAMGRSIKVQFQSGGDNSPAVYLLDGLRAQDDYNGWDINTPAFEWYYQSGLSVIMPVGGQSSFYSDWYSPACGKAGCTTYKWETFLTSELPQWLSANRSVKPTGSAAVGISMAGSSALILSVYHPQQFIYAGSLSALMDPSQGMGPSLIGLAMGDAGGYKASDMWGPSSDPAWQRNDPSLHIPELVANNTRLWIYCGNGTPSELGGANVPAEFLENFVRSSNLKFQDAYNAAGGHNAVFNLDANGTHSWEYWGAQLNAMKGDLQASLGAR